(19) United States
(12) Patent Application Publication
(10) Pub. No.: US 2017/0035347 A1
(43) Pub. Date: Feb. 9, 2017

(54) METHOD FOR MONITORING PREGNANCY IN MAMMALS

(71) Applicant: MB Device LLC, Louisville, KY (US)

(72) Inventors: Divya Cantor, Louisville, KY (US); Barry Simon, Lexington, KY (US); David Phelps, Louisville, KY (US); Vasiliy Abramov, Louisville, KY (US); John Vanderburgh, lLouisville, KY (US)

(73) Assignee: MB Device LLC, Louisville, KY (US)

(21) Appl. No.: 15/333,248

(22) Filed: Oct. 25, 2016

Related U.S. Application Data
(63) Continuation-in-part of application No. 13/611,265, filed on Sep. 12, 2012.

(51) Int. Cl. A61B 5/00; A61B 5/145; A61D 17/00; A61B 5/053; A61B 5/01

(52) U.S. Cl. CPC A61B 5/435; A61B 5/0011; A61B 5/053; A61B 5/01; A61D 17/008; A61B 5/6875; A61B 5/746; A61B 5/14539

(57) ABSTRACT

The present invention relates to a method for long term, continuous, monitoring of pregnant mammals particularly to detect the onset of labor. For The example, the method enables communication of data representing impedance through tissue of a cervix of a pregnant mammal an external module for display and monitoring of the collected data by a clinician.

### FIELD OF THE INVENTION

This invention relates to medical monitoring in general and more particularly to a system for monitoring pregnancy.

### BACKGROUND OF THE INVENTION

The birthing process in mammals has a normal or average duration, for example, in humans gestation is normally 266 days while for horses it normally ranges between 335 and 345 days. For cattle gestation averages 283 days while for swine about 113 to 116 days and for sheep 144 to 151 days. The actual period for human gestation is variable but is considered full-term to conclude between 38-42 weeks post-conception. In humans, for example, labor and birth that commences earlier than 36 weeks as a result of a high risk pregnancy or other complications presents serious if not fatal problems for the newborn infant that can well continue on to adulthood. Delivery at 30-32 weeks normally necessitates considerable time in an NICU and delivery very early at around 23 weeks is considered almost uniformly fatal.

For most domestic animals entering into labor and the birthing process occurs with little assistance. However, for humans in particular and certain valuable domestic animals, such as Thoroughbred horses, the gestation period in the final trimester is normally closely monitored for the beginning of labor and for complications such as preterm labor resulting in preterm birth.

For domestic animals a preterm birth will usually produce a still born or a weak and sickly animal requiring expensive attention. Especially in the case of Thoroughbred horses where stud fees may run up to six figures, a premature foal may not survive or perform to the expectations of the breeder resulting in a substantial financial loss.

For humans the societal burden of preterm births in the United States was estimated to be $26 billion in 2005 by the Committee on Understanding Premature Birth and Assuring Healthy Outcomes, under the auspices of the US Institute of Medicine. These costs include medical care services at $16.9 billion; maternal delivery costs $1.9 billion; early intervention services $611 million; special education services associated with a high incidence of disabling conditions associated with premature infants added $1.1 billion. Finally, the committee estimated that lost household and labor market productivity associated with those disabling conditions contributed $5.7 billion.

High-risk pregnancies, that is pregnancies that are suspected of presenting a serious adverse outcome in the mother and/or the baby, are not always detected early nor easily determined in advance so that preventative measures can be taken to avoid a preterm birth. Women pregnant for the first time or who may not have access to medical help can present a high-risk pregnancy without being aware of their condition. Early identification of high-risk pregnancies facilitates monitoring and prompt initiation of therapy. Early initiation of therapy has the potential to decrease the onset of labor, thus reducing the complications endured by the newborn as well as reducing the cost burden. However, monitoring of even normal pregnancies in the last trimester is also recommended if only to avoid unnecessary trips to the hospital because of false labor.

It is recommended that monitoring of a pregnancy, especially in the last half of the term of the pregnancy, be conducted frequently so that complications can be identified as well as the onset of labor in its very early stages. In the case of domestic animals monitoring of the animal's pregnancy will normally require the on-site presence of a veterinarian which may not be convenient.

In the United States and other Western countries where medical services are more readily available, a pregnancy may be monitored at the doctor's office as an outpatient or in the hospital. However, this requires periodic visits to the doctor's office or the hospital in order to monitor the patient's progress. In developing countries it may not be possible for a woman to see a doctor on a regular basis so that intervention to alleviate or treat a pregnancy related condition may be too late.

Various systems have been put forward to monitor the status of a pregnancy. For example U.S. Pat. No. 6,270,458 describes a system employing ultrasound to monitor the dilation of the cervix and descent of the presenting part during labor. This system employs small ultrasound reflectors disposed on either side of the cervical os and on the fetal presenting part to reflect ultrasound signals transmitted by an ultrasound interface unit located outside of the body.

Measurement of uterine electrical activity, electrohysterography, has been proposed as an alternative approach to the detection of labor. For example WO 94114373 (Garfield) records signals from embedded electrodes, WO 95/31932 and WO 96/39931 (Garfield) disclose a method which stores data and compares activity and U.S. Pat. No. 5,373,852 (Harrison) uses radiotelemetric transmission for sensing pressure, temperature and electrical activity. Rosenberg (WO 97/25922) discloses a further method for analysis of electromyographic data. These techniques relate exclusively to clinical settings, usually intrusive systems for data collection. U.S. Pat. No. 6,823,211 discloses a nonintrusive device including recording electrodes, a digital converter and a display unit that can be worn on the exterior of the abdomen or vaginal area of a body for the analysis of uterine electrical activity to monitor the progress of, and/or diagnose active labor and can be used to obtain an indication of uterine preparedness for labor in the initial phase of parturition before onset of active labor. Another system that relies on the measurement of uterine electrical activity is disclosed in U.S. Pat. No. 6,290,657 that, similar to the '211 patent, uses surface electrodes and a monitor to sense and analyze electrical signals that are transmitted by telephone to the physician's office.

The human birthing process is divided into four stages. Stage 1 occurs from the onset of labor to full cervical dilation. Stage 2 is from full cervical dilation to delivery; stage 3 from delivery to expulsion of placenta and stage 4 from expulsion of the placenta to afterbirth recovery. Thus, monitoring the physiological parameters of the cervix in humans and mammals allows the clinician to follow the progress of a pregnancy during stage 1 of the process where complications, particularly preterm labor, can be recognized and possibly treated. In that connection some successful work has been done in this area but the methods employed require the presence of the clinician and usually employ invasive procedures.

For example, in Patent Application Publication US 2009/0137925 the pregnancy is monitored by attaching a clip carrying a pair of tetra-pole electrodes to the patient's cervix. The electrodes that are electrically connected to a transducer cable that supplies the signal to an interface unit and monitor. The transducer cable carries analog to digital conditioning hardware while the interface unit includes an algorithm for comparing a typical impedance value with the impedance value of the cervix being measured. The resulting output can be a measure of time of labor vs. cervical stromal impedance (CSI) value or time in labor vs. cervical dilation and/or effacement for plotting a Friedman labor curve.

The prior art methods are inconvenient and are not suited for long term, continuous monitoring of the patient. In many cases monitoring involves the intervention of a clinician that requires the patient to visit the hospital or the clinician's office frequently to undergo the monitoring procedures. Placing sensors externally may introduce discrepancies in the data collected. For example, discrepancies due to variations in skin temperature and variations in contact pressure between the skin and the sensor. Other methods require that relatively large devices be placed in the patient that requires frequent removal to avoid odor and possible infection. Several methods require connection by wire to sensors at one end and to burdensome equipment at the output end that requires that the patient be non-ambulatory.

In addition, many prior art systems are concerned only with the measurement of vaginal contractions which can be an unreliable indicator of the onset of labor.

### SUMMARY OF THE INVENTION

The present invention relates to a method for long term, continuous, monitoring of pregnant mammals particularly to detect the onset of labor. For example, the method enables communication of data representing impedance through tissue of a cervix of a pregnant mammal an external module for display and monitoring of the collected data by a clinician.

The method of the invention comprises:
a. positioning at least one pair of electrodes in intimate contact with the patient's cervix;
b. applying alternating current to the cervix and measuring the potential through the tissue of the cervix;
c. converting the potential measurements to digital data points;
d. downloading the digital data points to an external module; and
e. correlating the digital data points representing potential measurements to impedance data points; and
f. recording and displaying the correlated data points to determine trends representative of physiological changes in the tissue of the cervix.

Analysis of electrical impedance of the tissue of the cervix provides the clinician with an indication of changes taking place in the cervix tissue before such changes are detected by conventional palpitation or measurement of muscle activity. In addition the method of the invention permits continuous monitoring of the patient by a clinician without the need for the patient to be physically present.

Accordingly, it is an object of the invention to monitor and record the physiological condition of cervix tissue from sensors placed immediately adjacent to and in intimate contact with a cervix.

An object of the invention is to provide a method for continuous monitoring of the condition of cervix ti s sue while permitting the subject to be completely ambulatory and have complete freedom of action.

Another object of the invention is to detect early changes in a cervix during pregnancy by the trend of digital data points representing impedance through the cervix tissue.

Yet another object of the invention is to provide a method for monitoring a normal pregnancy and to predict the onset of the birthing process.

Another object of the invention is to detect the early onset of preterm labor.

These and other objects of the invention from the following description of the invention taken in conjunction with the drawings in which:

### BRIEF DESCRIPTION OF THE DRAWINGS

FIG. 1 is a block diagram of the method of the invention;

FIG. 2 is an isometric view of an embodiment of an electrical impedance sensing device in accordance with the invention for securing electrodes in intimate contact with the tissue of the patients cervix;

FIG. 3 is a cross sectional view of the sensing device of FIG. 2 positioned on the cervix;

FIG. 4 is another embodiment of a sensing device adapted to be stitched to the patient's cervix;

FIG. 5 is another embodiment of a sensing device with an attached compartment for power and electronics;

FIG. 6 is a sectional view showing the sensing device of FIG. 5 placed in a patient;

FIG. 7 is another embodiment of a sensing device used in the method of the invention

FIG. 8A and FIG. 8B illustrate the operation of the method of the invention.

FIG. 9 is a plot of impedance data taken at high frequency from a sensing device worn continuously for five days.

### DESCRIPTION OF THE INVENTION

The method of the invention is suited for monitoring the pregnancy of mammals, however for ease of description the invention will be described in connection with monitoring the pregnancy of a human female.

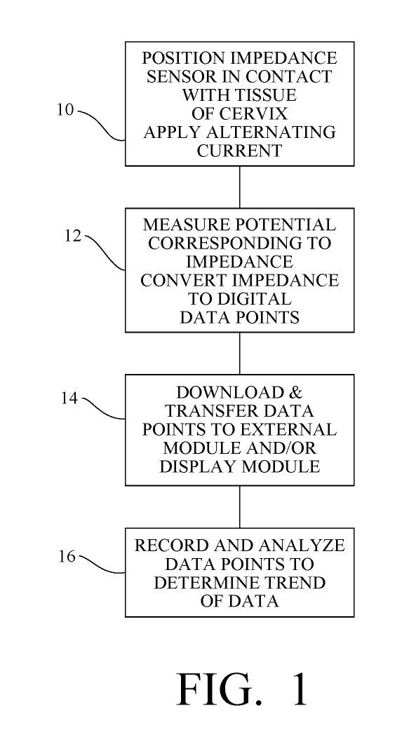

FIG. 1 illustrates generally the steps of the invention in which at step 10 a sensing device comprising at least one pair of electrodes are positioned in intimate contact with the patient's cervix. An alternating current is applied to the cervix and potential that can be correlated to electrical impedance through the tissue of the cervix is measured at one or more frequencies. The measurements are converted by suitable circuitry in the sensing device to digital data points at 12. In step 14 the data points are transmitted, preferably wirelessly, to an external module located on or near the subject being monitored for recording. The external module may directly display the data points, such as when it is a computer, or may be an interface/telemetry device which collects and subsequently transmits the data to a display module. Because the sensing device and the external module are normally in relatively close proximity, communication between the sensing device and the external module is most conveniently achieved using a conventional communication system such as Bluetooth® or radio. In step 16 the digital data points are correlated to impedance measurements and are recorded, displayed and analyzed to determine trends representative of physiological changes in the tissue of the cervix. Downloading to the external module may be accomplished manually by transferring the digital data points on a flash drive or similar device where the external module is more remote, the data points may be wirelessly downloaded by suitable means, such as for example by radio frequency.

The method of the invention is designed as a convenient method for continuously monitoring a pregnancy without the inconvenience of having the patient visit a clinic or hospital in order for the clinician to examine the patient. The patient can be continuously monitored to determine the physiological condition of the cervix tissue at any time during the pregnancy, particularly during the third trimester where high risk pregnancies can result in preterm birth.

Impedance through cervix tissue is an indicator of physiological condition of the tissue. Thus, trends in impedance data can indicate critical physiological changes in cervix tissue that signal the onset of labor before changes in effacement and ripeness can be detected by conventional techniques. This particularly critical in high risk pregnancies where steps can be taken early on to delay a preterm birth and reduce danger to the infant and the mother.

Impedance is correlated from potential measurements from alternating current applied to the cervix by a conventional algorithm based on Ohm's Law. Potential may be measured at various frequencies depending on the depth of tissue being monitored. Thus, at low frequency, about 35 Hz for example, correlated impedance monitors the outer or surface area of the tissue. At medium frequency, about 5 KHz, the correlated impedance monitors a deeper area of tissue. At high frequency, about 100 KHz for example, correlated impedance monitors the physiological condition of the deepest tissue. In practice it is recommended that measurements be taken at low, medium and high frequency.

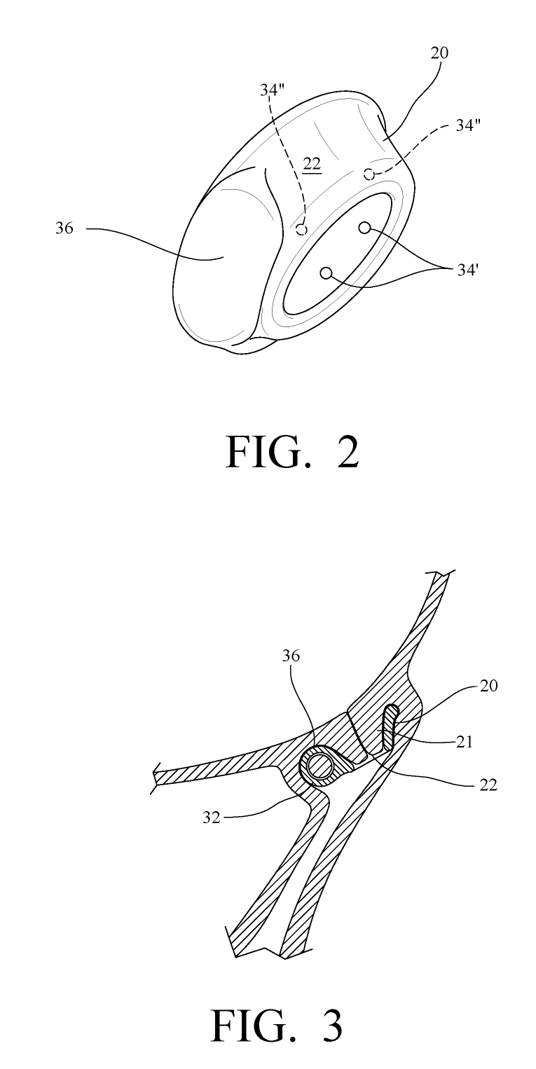

FIG. 2 illustrates the first step of the method of this invention with the sensing device 20 in position on the patient's cervix 21 (FIG. 3). As illustrated in this embodiment, the sensing device 20, comprises an annular body 22 that includes two pairs of the electrodes 34′ and 34″ on the inner radial surface of the annular body for contact with the cervix 21. One pair of electrodes 34 is referred to as the injecting electrodes for introducing an alternating current to the cervix tissue and the second pair 34″, referred to as measuring electrodes, measures potential the through the cervix tissue. The choice of which electrode pair, 34′ or 34″, is utilized as the injecting electrodes is not critical. The annular body 22 also contains a pocket 36 in which are sealed a battery power supply (not shown), a chip for receiving and converting the signals from the electrodes into digital data points (not shown) and the associated circuitry (not shown) for downloading and transmitting the digital data points.

As shown in FIG. 2 and FIG. 3, two pairs of electrodes are illustrated. It will be understood, however, that a single pair electrodes (FIG. 5) can serve both as the injecting electrodes and the potential sensing electrodes. Also, more than two pair of electrodes may be utilized for increased accuracy of the impedance measurements such as illustrated in FIG. 7.

It will be understood that contact between the electrodes 34′ and 34″ and the cervix 21 of a patient must be substantially uniform during the monitoring process. Thus the location of the electrodes 34′ and 34″ on the cervix 21 and the amount of pressure applied to the electrodes against the cervix will affect the measured impedance. A change in the location and/or the applied pressure during monitoring will cause a change in impedance measurements and affect the trend of data. Accordingly, the annular body 22 comprises a flexible, resilient, non-conductive biocompatible material, such as polyurethane, silicone, silicone rubber or the like, that can be slightly stretched for placement around the cervix 21. Once in place the stretching force is removed and the annular body 22 returns to its original diameter to provide a secure, intimate contact between the electrodes 34′ and 34″ and the surface of the cervix 21. As long as the annular body 22 remains in its original position around the cervix 21, the applied pressure on the electrodes 34′ and 34″ and their position on the cervix will remain constant.

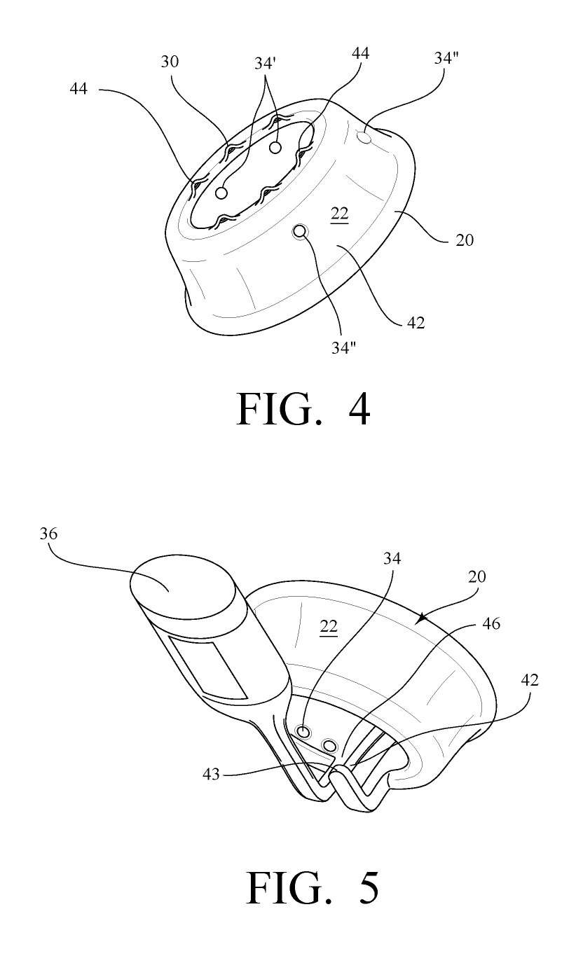

In view of the fact that the patient is ambulatory during the monitoring process there exists the danger of a shift in the position of the annular body 22 on the cervix 21 which will result in the change of the position of the electrodes 34. Any such change can also affect the applied pressure on the electrodes. Either or both events will change the resulting data which can give a false indication of a change of impedance in the cervix tissue or could hide an impedance change in cervix tissue. Notice of any such shift the annular body 22 can be indicated from an unusual change in the total electrode potential which is the sum of the potential measured between the injecting electrodes 34′ and the pair of electrodes 34″ that are measuring cervix tissue potential. Such an unusual change in the total potential correlates to an unusual change in total impedance and will clearly show in the displayed data trend. Such a change in the data trend may also be an indication of a system malfunction. As a precaution, however, depending on the predicted activity of the patient, the sensing device 20 can be adapted for stitching on to the cervix 21 to prevent any shifting. Referring to FIG. 4 the sensing device 20 is provided with an annular skirt 28 with several stitching eyes 30 for securing the sensing device 20 to the patient's cervix 21.

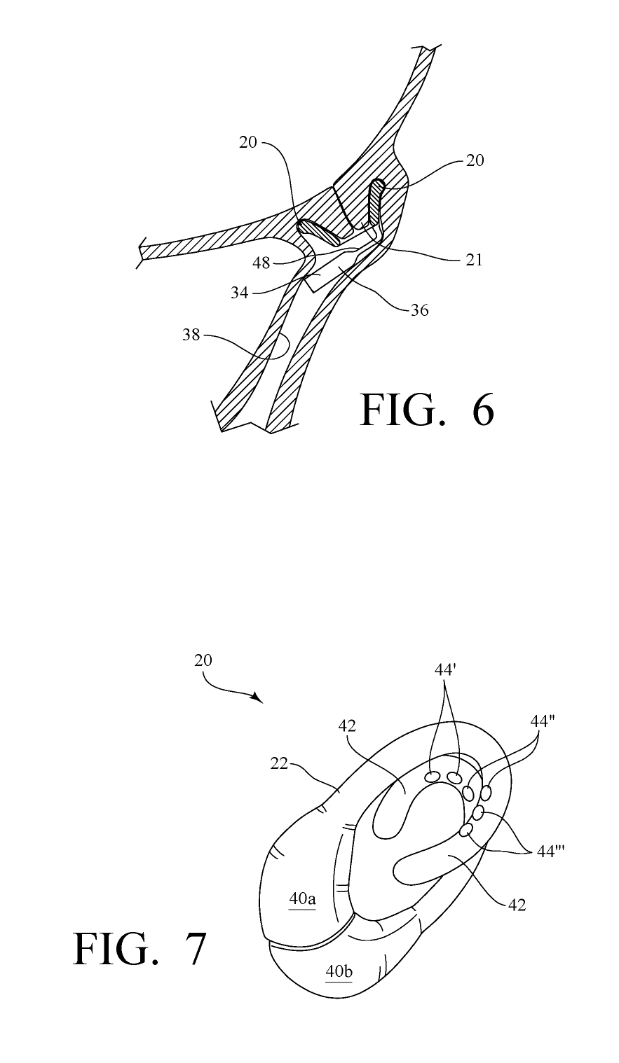

Yet another embodiment of the sensing device, shown generally as 20, is illustrated in FIG. 5 and FIG. 6 in which the annular body 22 is interrupted to define free ends 42 and 43. Free end 42 extends away from the annular body 22 and is enlarged to define a compartment 36 for a battery and associated electronics. A single pair of electrodes 34 serves both as the injecting electrodes and the measuring electrodes. As shown in FIG. 6, which is partially in section, the free end 43 is sufficiently flexible to allow the compartment 36 to lie in the vagina 38 below the cervix 21.

In another embodiment shown in FIG. 7 the sensing device 20 is configured as a constricting pessary to maintain closure of the cervix 21. In this embodiment the sensing device 20 comprises the annular body 22 on which is formed enlarged compartments 40a and 40b in which are respectively disposed the battery and the associated electronics. The annular body 22 is interrupted to define a pair of resilient, flexible U-shaped arms 42. In this embodiment three electrodes are disposed in each of the arms to define three electrode pairs 44′, 44″ and 44′″. One pair of electrodes 44″ comprises the injecting electrodes and electrode pairs 44″ and 44′″ comprise the impedance measuring electrodes. As stated above it is not critical which pair of electrodes comprise the injecting electrodes.

The sensing device 20 is positioned around the cervix by spreading the U-shaped arms 42. Once positioned, the arms 42 are allowed to return to their original configuration so that each arm securely contacts the cervix to provide constrictive support of the cervix as a pessary and to maintain the electrode pairs 44′, 44″ and 44′″ securely positioned in intimate contact with the cervix.

It will be understood that monitoring of the physiological parameters of a pregnancy may require that body temperature and pH also be monitored and recorded. The various embodiments of the sensing devices described herein may also be provided with the appropriate sensors for detecting body temperature and pH. These sensing devices are well understood in the art and do not per se form a part of this invention. For example pH can be measured by ion sensitive field effect transistors (ISFET) and miniaturized electronic temperature sensors for converting pH and temperature signals to digital data and holding this data in separate packets for transmission to the external module for inclusion in a data base.

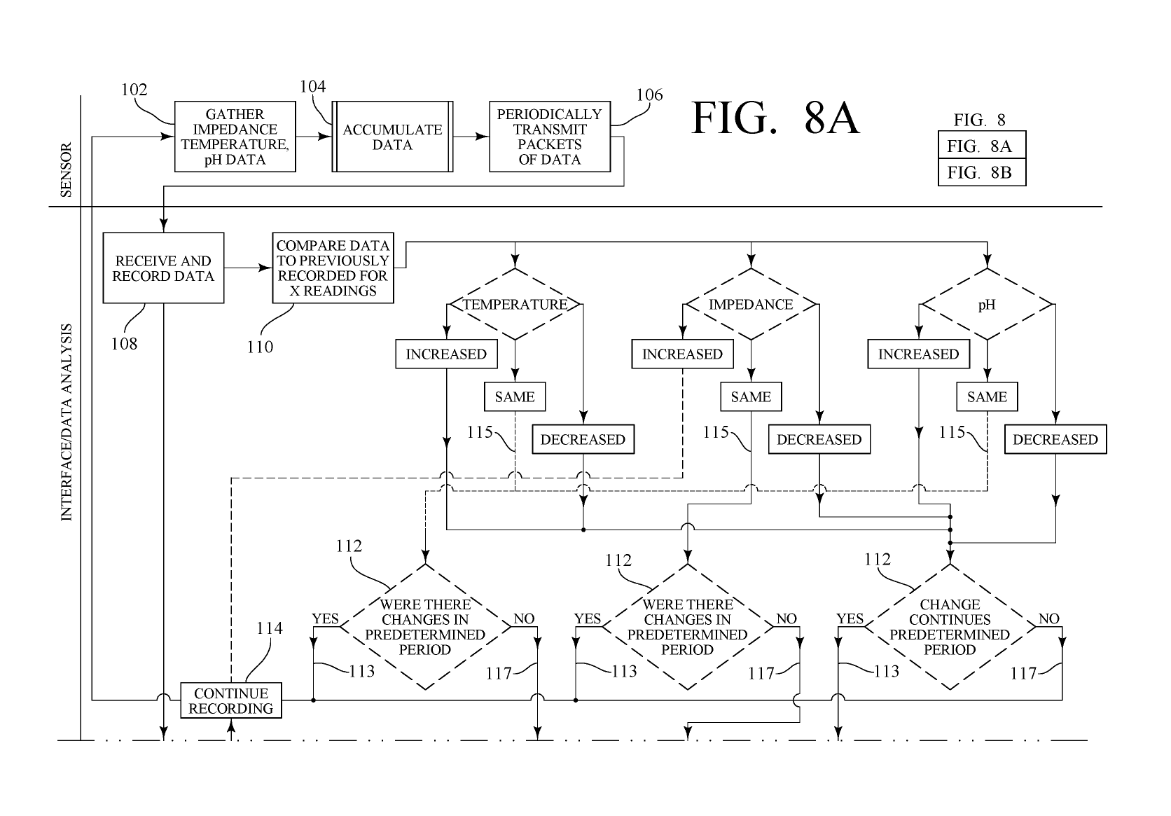

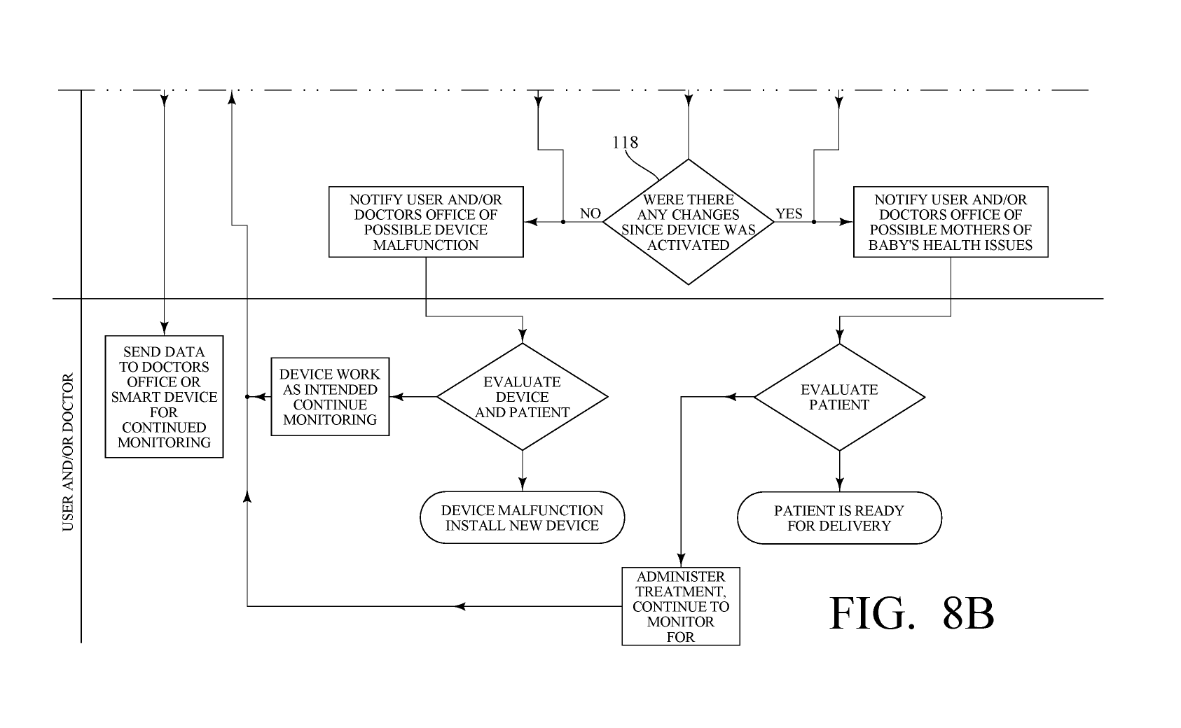

FIGS. 8A and 8B) is a schematic illustrating in detail the method of the invention. Once positioned in intimate contact with the patient's cervix, the sensing device 20 in step 102 gathers potential measurements and optionally pH and temperature measurements and converts the measurements into digital data representing the physiological condition of the tissue of the cervix. The data are accumulated at the sensing device 20 at step 104 and periodically downloaded to an external module as a packet of each of the physiological parameters of impedance, pH and temperature, in step 106. In step 104 the data may be accumulated at the sensing device 20 over a period ranging up to several hours and downloaded to the external module as a packet to conserve battery life. The data packets are downloaded wirelessly by any convenient wireless transmission technique such as Bluetooth®, Rf or infrared.

The external module receives and records the transmitted data in step 108. Periodically the recorded data is downloaded to a remote display module, such as a computer, where the potential measurements are correlated to impedance for display of the newly received data along with previously received data to show a trend of impedance measurements, and optionally pH and temperature, over time. Data representing impedance and optionally temperature and pH for each transmitted packet of data can be analyzed by being compared either to a data base consisting of typical values or to a data base consisting of previously recorded data for the individual at step 110 to determine an increase, no change or decrease in the trend of the data values.

If, after comparing the newly received packet of data with the database for any or all of the measured parameters of impedance, temperature and pH to develop a trend, there is no substantial change in the trend, monitoring steps 104 through 110 will continue. In step 112 a change in the trend of the data values for any or all of the parameters of impedance, temperature and pH for each transmitted packet of data over a predetermined period, for example 8 hours, can indicate an impending change in the cervix and will trigger a signal 113 to the user and the clinician to more closely monitor data output or to personally examine the individual. In the meantime monitoring and recording of the parameters is continued at 114. Thus, for example, a change in the trend of data for impedance measured through the tissue of the cervix 21 may predict a future change in cervix effacement, ripeness or dilation indicating the onset of birth or change in the health of the subject being monitored. In the case of impending preterm, birth steps may be taken to delay the birthing process and thus allow for further development of the fetus. Likewise, a trend showing a change over time in pH and or temperature may indicate infection or other health issue that can be treated by the clinician.

In the event there are no substantial changes in the trend of a received data packet for a parameter, signal 115 issues and the data is reviewed at 112 for changes that have been recorded over the predetermined time. If changes have been recorded during this period signal 113 is issued and monitoring continues. If the recorded data for the parameter is a flat line for the predetermined period, a signal 117 will be issued instructing at 118 that all recorded data should be reviewed for data changes from the time the device was activated. If there has been no change of data, a signal is transmitted to the external module notifying the patient and the clinician of a possible device malfunction. The sensing device 20, the external module and the patient can be checked to determine source of the problem.

Time periods for transmitting data packets to the external module are programmed in the electronics of the sensing device 20. As mentioned above, it is preferred to keep transmission of the packets of digital data to a minimum, particularly at the earlier stage of pregnancy, to preserve battery life. At a later stage or critical period during the pregnancy the period between data packet transmission may be shortened. Likewise, the predetermined period for data review at 112 is programmed into the electronics of the external module. Preferably this period can be programmed by the clinician and will be adjusted depending on the parameter being monitored and the stage of the pregnancy. For example, in the early stage of a pregnancy the predetermined period for impedance measurement may be as long as several months. In the later stages, however, change in impedance becomes more critical and the predetermined period may be a few hours.

The data can be displayed by the interface device 14 to provide a read out of any changes or lack thereof from the previously recorded data. Thus, those attending the individual being monitored, or in the case of a human, the patient herself, will receive immediate notification of a problem with the pregnancy or a possible device malfunction. The compared data and data base may be periodically wirelessly transmitted in step 111 through the transmitter 16 to the monitor 18 that may be remotely located for display and monitoring by the clinician. In addition the data can be plotted on a curve, along with the previously recorded data. Alternatively, only a signal indicating a significant change in one or all of the parameters being monitored or a problem with the sensing device 20 or the external module will be transmitted to the monitor 18 to alert the clinician to a potential problem that may require attention.

For larger clinical operations the remote monitor may be a central server for receiving data from several patients. In this case the data may be monitored by a single individual who then notifies the patient's clinician in the event that patient's data trend indicates a significant change or the server itself can signal the patient's clinician.

### Example

The proof of concept was demonstrated as follows. A non-pregnant female volunteered to wear a sensing device of the type described in connection with FIG. 2 continuously for a period of 5 days. Her activities were not restricted and in all respects she performed her normal activities. The only limitation was that she kept an interface/telemetry device on her person during the period she wore the sensing device.

The impedance was measured at 100 KHz and the measurements were converted in the sensing device into digital data points. The data points were transmitted from the sensing device to the interface/telemetry device in 15 minute packets and downloaded to a computer every 12 hours.

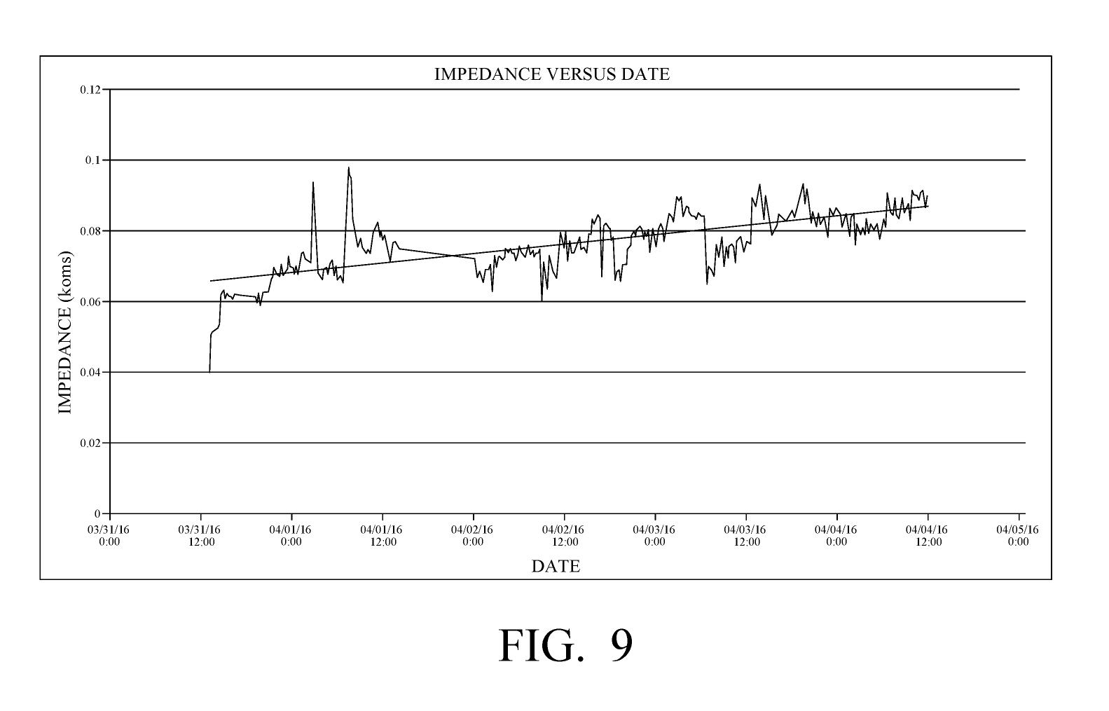

FIG. 9 is a plot of Impedance versus Date. The data from April 2 to the end of the test period was relatively uniform and the trend line was essentially flat. This was expected since the subject was not pregnant and in good health. There was a wild data swing during April 1. The data swing occurred because the subject was not wearing the sensing device correctly. This was corrected and the data trend settled down. From this experience it is clear that any problems with the sensing device or the system will become apparent and can be corrected.

While various embodiments have been described above, it should be understood that they have been presented by way of example only, and not limitation. Thus, the breadth and scope of a preferred embodiment should not be limited by any of the above-described exemplary embodiments, but should be defined only in accordance with the following claims and their equivalents.

The invention claimed is:

1. A method for long term, continuous monitoring of pregnant mammals by the measurement of physiological parameters of the cervix, said method comprising the steps of:
a) placing at least one pair of electrodes in intimate contact with the cervix of a female mammal;
h) applying alternating current to the tissue of said cervix through at least one pair of electrodes;
c) maintaining said at least one pair of electrodes in intimate contact with the cervix during said continuous monitoring of said female mammal;
d) sensing potential through the tissue of said cervix by said at least one pair of electrodes;
e) converting said potential to digital datapoints; and
f) downloading and transmitting said digital data points to an external module for correlation of potential to impedance and recording of said impedance data points to determine trends representative of the physiological condition of the cervix tissue.

2. The method of claim 1 wherein said potential through said cervix tissue is measured at selected frequencies depending on the depth of cervix tissue being monitored.

3. The method of claim 1 wherein said potential through said cervix tissue is measured at a low frequency.

4. The method of claim 1 wherein said potential through said cervix tissue is measured at a medium frequency.

5. The method of claim 1 wherein said potential through said cervix tissue is measured at a high frequency.

6. The method of claim 1 wherein said potential through said cervix tissue is measured at 35 Hz.

7. The method of claim 1 wherein said potential through said cervix tissue is measured at 5 kHz.

8. The method of claim 1 wherein said potential through said cervix tissue is measured at 100 kHz.

9. The method of claim 1 wherein said potential through said cervix tissue is measured at low, medium and high frequencies.

10. The method of claim 1 wherein at least two pairs of electrodes are placed in intimate contact with the cervix, one pair of said at least two pairs of electrodes serving as injecting electrodes for applying alternating current to the tissue of said cervix.

11. The method of claim 1 further including a sensing device having a surface for intimate contact with said cervix, said at least one pair of electrodes being disposed on said surface for intimate contact with said cervix, said sensing device incorporating a power supply, a chip for receiving and converting signals from said at least one pair of electrodes into digital data points and circuitry for downloading and transmitting said digital data points.

12. The method of claim 10 wherein said sensing device comprises an annular device defining an inner radial surface for contact with said cervix, said at least one pair of electrodes being disposed on said inner radial surface and being in electrical communication with said power supply and said chip, said annular body comprising a flexible, resilient non-conductive material for maintaining said inner radial surface and said at least one pair of electrodes in intimate contact with said cervix.

13. The method of claim 10 further including an interface telemetry device for receiving digital data points from said sensing device and for transmitting said digital data points to said external module.

14. The method of claim 1 wherein said digital data points are downloaded and transmitted to said external module in packets at selected time periods.

15. A method for long, term, continuous monitoring of pregnant mammals by the measurement of physiological parameters of the cervix, said method comprising the steps of:
a) placing at least two pairs electrodes in intimate contact with the cervix of a female mammal, at least one pair functioning as injecting electrodes and at least one pair as measuring electrodes;
b) applying alternating current to said cervix tissue through said at least one pair of injecting electrodes;
c) maintaining at least two pairs of electrodes in intimate contact with said cervix during said continuous monitoring of said female mammal;
d) pleasuring impedance through said tissue of said cervix and creating an electrical signal responsive to the strength thereof by said at least one pair of measuring electrodes;
e) converting electrical signal to digital data;
f) transmitting said digital data to an external display module;
g) correlating said digital data from potential measurements to impedance measurements, analyzing said digital data by comparing said digital data with a database representative of physiological parameter being sensed to determine a change in data trend;
h) recording, said digital data;
i) continuing said sensing, converting, transmitting, correlating and comparing digital data throughout said long term continuous monitoring period;
j) transmitting an alarm signal in the event of a significant change in the trend of said digital data; and
k) transmitting an alarm signal in the event of device malfunction.

16. The method of claim 15 wherein at predetermined time intervals said digital data is transmitted to a remote display module.

17. The method of claim 15 wherein said remote display module is a central server that collects, analyses and stores data from multiple individuals and issues notification to clinicians responsible for each individual.

18. The process of monitoring a pregnancy for early detection of conditions characteristic of premature birth, which comprises:
a) detecting the physiological parameters of cervical tissue impedance, pH and temperature and generating an electrical signal proportional to the intensity of each parameter;
b) converting said electrical signal to a digital data point;
c) transmitting said digital data point to an external module;
d) comparing said digital data point with previously recorded data points representative each said physiological parameter being sensed;
e) adding said digital data point to previously recorded digital data points for each said physiological parameter being monitored to develop a trend;
f) continuing said sensing, converting, transmitting and comparing with a base of the physiological parameters throughout a predetermined period; and
g) transmitting an alarm signal to a monitor in the event a significant change in a trend of said digital point.
